(19) United States
(12) Patent Application Publication
(10) Pub. No.: US 2011/0306152 A1
(43) Pub. Date: Dec. 15, 2011

(54) System and Method for Determining Concentration of a Predetermined Osteoarthritis Biomarker in a Urine Sample

(75) Inventors: Chi-Yen Shen, Dashu Township (TW); Chih-Hsin Hung, Dashu Township (TW); I-Fen Chen, Dashu Township (TW); Shih-Han Wang, Dashu Township (TW)

(73) Assignee: I SHOU UNIVERSITY, Dashu Township (TW)

(21) Appl. No.: 12/797,433

(22) Filed: Jun. 9, 2010

(51) Int. Cl. G01N 33/564; G01N 33/48

(52) U.S. Cl. 436/506; 422/68.1

(57) ABSTRACT

A system for determining concentration of a predetermined osteoarthritis biomarker in a urine sample includes first cell and first limiting elements, a quartz crystal microbalance (QCM) sensor device having a sample contacting side, and a monitoring device.

The first cell element has a sensor confronting side formed with a recess portion. The first limiting element is disposed at the sensor confronting side, is disposed to surround the recess portion, contacts the sample contacting side, and cooperates with the recess portion and the sample contacting side to confine a sample receiving space for receiving the urine sample.

The QCM sensor device includes a quartz resonator and an antibody applied to the quartz resonator and capable of binding with the biomarker, generates a concentration signal corresponding to mass of the biomarker that binds to the antibody, and is coupled to the monitoring device to provide the concentration signal thereto for processing thereby.

### BACKGROUND OF THE INVENTION

1. Field of the Invention

The present invention relates to osteoarthritis detection, more particularly to a flow sensor assembly suitable for use in a system and method for determining concentration of a predetermined osteoarthritis biomarker in a urine sample.

2. Description of the Related Art

An increasing number of societies have reached a state of aging society. Consequently, influence of aging-related diseases has become significant and has been gaining attention. In orthopedics, a high proportion of elderly patients suffer from degeneration problems or diseases of the joints. Conventional clinical treatments of orthopedic degeneration problems and diseases involve conservative prescription of anti-inflammatory drugs and physiotherapy in order to alleviate pain. As for patients who suffer from severe orthopedic degeneracy, conventional clinical treatment often resorts to surgery to install artificial joints. Therefore, it is important to provide a means for diagnosing joint degeneration and diseases at an early stage of their development.

Currently, diagnosis of orthopedic degeneracy and diseases is performed with the use of X-ray, magnetic resonance imaging (MRI), arthroscopes, or a combination thereof. Although such diagnosis methods have been proven to be feasible, adverse diagnosis outcomes thus obtained usually indicate irreversible joint impairment as a result of late or no treatment.

U.S. Pat. No. 6,642,007 discloses a method that employs the enzyme-linked immunosorbent assay (ELISA) for diagnosing rheumatoid arthritis (RA) or osteoarthritis (OA). In the method, a monoclonal antibody or a polyclonal antibody are used to capture type II collagen fragments or type II collagen neoepitope (TIINE) in a urine sample. Although such method is practicable, it requires pre-processing of the urine sample, such as dilution, sedimentation, and centrifugation, followed by purifications and modifications of proteins, which can be a very time-consuming process.

### SUMMARY OF THE INVENTION

Therefore, an object of the present invention is to provide a system for determining concentration of a predetermined osteoarthritis biomarker in a urine sample. Accordingly, a system according to the present invention includes a flow sensor assembly and a monitoring device.

The flow sensor assembly includes a quartz crystal microbalance (QCM) sensor device including a quartz resonator, and an antibody applied to the quartz resonator and capable of binding with the predetermined osteoarthritis biomarker in the urine sample. The QCM sensor device has a sample contacting side and generates a concentration signal corresponding to mass of the predetermined osteoarthritis biomarker that binds to the antibody.

The flow sensor assembly further includes a flow cell device including a first cell element that has a sensor confronting side formed with a recess portion, and a first limiting element that is disposed at the sensor confronting side of the first cell element, that is disposed to surround the recess portion of the first cell element, that contacts the sample contacting side of the QCM sensor device, and that cooperates with the recess portion of the first cell element and the sample contacting side of the QCM sensor device to confine a sample receiving space for receiving the urine sample. The monitoring device is coupled to the QCM sensor device for receiving the concentration signal therefrom, and is operable to determine concentration of the predetermined osteoarthritis biomarker in the urine sample using the concentration signal.

Another object of the present invention is to provide a flow sensor assembly for a liquid sample.

Accordingly, the flow sensor assembly of the present invention includes a sensor device and a flow cell device. The sensor device has a sample contacting side. The flow cell device has a sensor confronting side that is formed with a recess portion, and includes a limiting element that is disposed at the sensor confronting side, that is disposed to surround the recess portion, that contacts the sample contacting side, and that cooperates with the recess portion and the sample contacting side to confine a sample receiving space for receiving the liquid sample.

Yet another object of the present invention is to provide a flow cell device adapted for use with a sensor device to detect a liquid sample.

Accordingly, a flow cell device of the present invention includes a cell element and a limiting element. The cell element has a sensor confronting side formed with a recess portion. The limiting element is disposed at the sensor confronting side of the cell element, is disposed to surround the recess portion, and is adapted to contact a sample contacting side of the sensor device. The limiting element, together with the recess portion of the cell element, is adapted to cooperate with the sample contacting side of the sensor device to confine a sample receiving space for receiving the liquid sample.

Yet another object of the present invention is to provide a method for determining concentration of a predetermined osteoarthritis biomarker in a urine sample. The method includes the steps of:
a) providing a flow sensor assembly which comprises a quartz crystal microbalance (QCM) sensor device and a flow cell device,
the QCM sensor device including a quartz resonator and an antibody applied to the quartz resonator and capable of binding with the predetermined osteoarthritis biomarker in the urine sample, the QCM sensor device having a sample contacting side and generating a concentration signal corresponding to mass of the predetermined osteoarthritis biomarker that binds to the antibody,
the flow cell device including a cell element that has a sensor confronting side formed with a recess portion, and a limiting element that is disposed at the sensor confronting side of the cell element, that is disposed to surround the recess portion of the cell element, that contacts the sample contacting side of the QCM sensor device, and that cooperates with the recess portion of the cell element and the, sample contacting side of the QCM sensor device to confine a sample receiving space for receiving the urine sample;
b) with the use of a monitoring device coupled to the QCM sensor device, measuring a frequency response of the QCM sensor device prior to contacting the QCM sensor device with the urine sample;
c) supplying the urine sample into the sample receiving space so as to bring the sample contacting side of the QCM sensor device into contact with the urine sample;
d) removing the urine sample from the sample receiving space and supplying a buffer solution into the sample receiving space;
e) with the use of the monitoring device, measuring a frequency response of the QCM sensor device from the concentration signal after supplying the buffer solution into the sample receiving space; and
f) configuring the monitoring device to determine the concentration of the predetermined osteoarthritis biomarker in the urine sample with reference to the measured frequency responses obtained in steps b) and e).

The system for determining concentration of an osteoarthritis biomarker in a urine sample, according to the present invention, has a modular design, only requires a few operating steps and short diagnosis time, does not require pre-processing of the urine sample, and therefore is suitable for implementation as a portable device at a relatively low cost.

### BRIEF DESCRIPTION OF THE DRAWINGS

Other features and advantages of the present invention will become apparent in the following detailed description of the preferred embodiment with reference to the accompanying drawings, of which:

FIG. 1 is a schematic diagram illustrating the preferred embodiment of a system for determining concentration of a predetermined osteoarthritis biomarker in a urine sample, according to the present invention;

FIG. 2 is an enlarged schematic diagram illustrating the assembly relation of a flow cell device and a quartz resonator of a QCM sensor device of the preferred embodiment;

FIG. 3 is a circuit diagram of a Colpitts oscillator of a feedback oscillator unit of the QCM sensor device; and

FIG. 4 is a flowchart of the preferred embodiment of a method for determining concentration of a predetermined osteoarthritis biomarker in a urine sample, according to the present invention.

### DETAILED DESCRIPTION OF THE PREFERRED EMBODIMENT

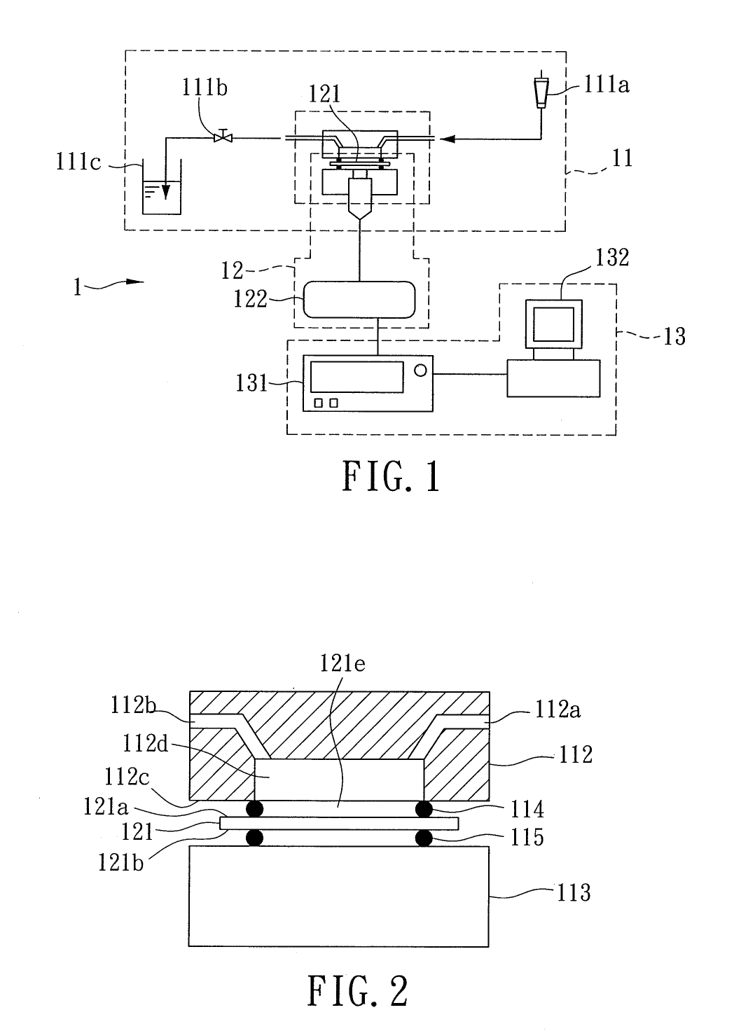

Referring to FIGS. 1 and 2, the preferred embodiment of a system for determining concentration of a predetermined osteoarthritis biomarker in a urine sample, according to the present invention, includes a flow sensor assembly 1 that includes a flow cell device 11 and a quartz crystal microbalance (QCM) sensor device 12.

The flow cell device 11 includes first and second cell elements 112, 113 made of a polytetrafluoroethylene material, and first and second limiting elements 114, 115. The QCM sensor device 12 includes a quartz resonator 121 including a quartz body that has a first side 121a serving as a sample contacting side and a second side 121b opposite to the first side 121a.

The first cell element 112 has a sensor confronting side 112c formed with a recess portion 112d. The first limiting element 114 is a waterproofing ring made of plastic, is disposed at the sensor confronting side 112c, contacts the sensor confronting side 112c, is disposed to surround the recess portion 112d, and cooperates with the recess portion 112d of the first cell element 112 and the first side 121a (i.e., the sample contacting side) of the quartz body of the quartz resonator 121 to confine a sample receiving space 112e for receiving the urine sample.

The second cell element 113 is disposed at the sensor confronting side 112c of the first cell element 112 and cooperates with the first cell element 112 to form a sensor disposing space within which the quartz resonator 121 is disposed. The second limiting element 115 is also a waterproofing ring made of plastic, is disposed between the quartz resonator 121 and the second cell element 113, and cooperates with the first limiting element 114 to enhance securing of the quartz resonator 121 to the first cell element 112 by the second cell element 113. It is to be noted that implementation of the first and second limiting elements 114, 115 is not limited to such.

The first cell element 112 is further formed with first and second channels 112a, 112b, each of which is in fluid communication with the recess portion 112d. The flow cell device 11 further includes first and second flow control units 111a, 111b, each of which is coupled to the first cell element 112 for controlling flow of the urine sample through a respective one of the first and second channels 112a, 112b. The first flow control unit 111a and the first channel 112a cooperate to form a supply path for introducing the urine sample into the sample receiving space 112e. On the other hand, the second flow control unit 111b and the second channel 112b cooperate to forma discharge path for discharging the urine sample from the sample receiving space. In the present embodiment, each of the first and second flow control units 111a, 111b consists of a tube having one end coupled to a respective one of the first and second channels 112a, 112b, and a valve for controlling liquid flow through the tube. However, implementation of the first and second flow control units 111a, 111b is not limited to such.

The quartz resonator 121 further includes first and second electrodes (not shown) that are disposed on the first and second sides 121a, 121b of the quartz body of the quartz resonator 121, respectively. The QCM sensor device 12 further includes an antibody (not shown) applied to the first electrode and capable of binding with the predetermined osteoarthritis biomarker in the urine sample. The quartz resonator 121 of the QCM sensor device 12 generates a frequency signal corresponding to the mass of the predetermined osteoarthritis biomarker that binds to the antibody.

The antibody is selected from the group consisting of an anti-type II collagen neoepitope (TIINE), an anti-cartilage oligometric matrix protein (COMP), a neoepitope at the C-terminus of the ¾ length type II collagen cleavage product created by the cleavage of type II collagen by collagenases, and combinations thereof.

In the present embodiment, the antibody is TIINE, and the first electrode is a gold electrode. The quartz resonator 121 has a frequency response ranging between 10 MHz and 20 MHz, a Q factor ranging between 100 and 120, and a diameter of 8 mm. The first and second electrodes have a diameter ranging between 3.6 mm and 4.5 mm. Furthermore, applying the antibody to the first electrode includes the steps of:
1) disposing the portion of the first electrode to which the antibody is to be applied in 2.5 mM of thioctic acid for 24 hours at room temperature (25° C.);
2) activating chemically the surface with 0.5 M of
   1-ethyl-3-(3-dimenthylaminopropyl)carbodiimide for three hours; and
3) dissolving the antibody (i.e., TIINE) in a phosphoric acid solution that has a pH value of 7.4, and bringing the first electrode into contact with the solution containing the dissolved antibody for 12 hours at room temperature.

However, applying the antibody to the first electrode may be implemented differently in other embodiments according to design need.

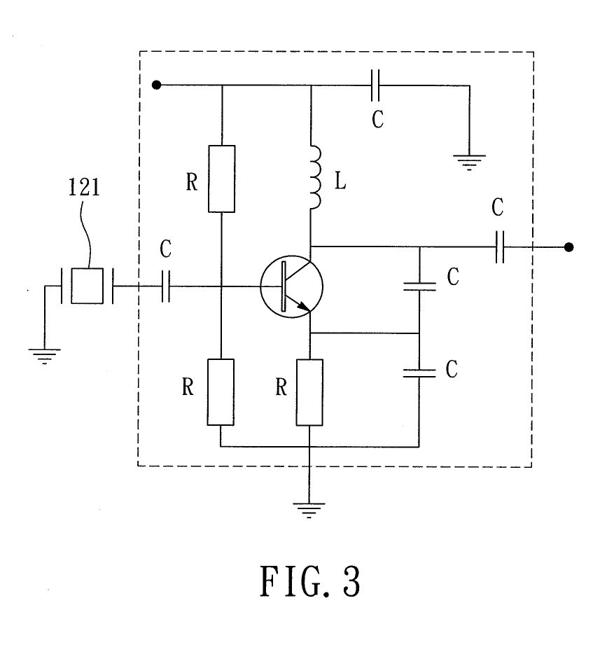

The QCM sensor device 12 further includes a feedback oscillator unit 122 constituted by a frequency-sensitive feedback circuit (see FIG. 3) coupled electrically to the first electrode of the quart z resonator 121 (the second electrode of the quartz resonator 121 is electrically grounded), and an active amplifier (not shown) coupled electrically to the frequency-sensitive feedback circuit.

Referring to FIG. 3, in the present embodiment, the frequency-sensitive feedback circuit is a Colpitts oscillator that is configured to operate in a frequency range covering the resonant frequency of the quartz resonator 121, that is pre-tuned to generate a driving signal with a frequency at which the quartz resonator 121 resonates, and that provides the driving signal to the quartz resonator 121 via the first electrode thereof so as to drive operation thereof. Since the structure and the tuning method of Colpitts oscillators are generally known by one who is skilled in the art, they will not be detailed hereinafter for the sake of brevity.

The quartz resonator 121 receives the driving signal, is operable to generate from the driving signal the frequency signal corresponding to mass of the antigens that are captured by the antibody, and provides the frequency signal as a feedback to the frequency-sensitive feedback circuit, which is further configured to adjust the driving signal to conform to the frequency signal in terms of frequency. That is to say, with the binding of the antibody and the antigens, the increased mass on the first side 121a of the quartz body lowers the resonant frequency of the quartz resonator 121, thereby forcing the frequency-sensitive feedback circuit to adjust the frequency of the driving signal to conform to that of the frequency signal.

The active amplifier receives the frequency signal from the frequency-sensitive feedback circuit and is operable to generate the concentration signal by amplifying the frequency signal in a manner that the frequency and concentration signals have substantially identical frequency and phase characteristics. The active amplifier is set to have a gain and a phase such that the frequency-sensitive feedback circuit and the active amplifier of the feedback oscillator unit 122 in combination have a gain greater than 1 and are substantially in-phase. Since the structure and the tuning method of active amplifiers are also generally known by one who is skilled in the art, they will not be detailed hereinafter for the sake of brevity.

The system of the preferred embodiment further includes a monitoring system 13 including a signal analyzer 131 and a processing unit 132. In the present embodiment, the signal analyzer 131 is a frequency counter coupled electrically to the feedback oscillator unit 122 for measuring frequency of the concentration signal. The processing unit 132 is a personal computer (PC) coupled to the signal analyzer 131 and configured to execute an algorithm, which is based on a mathematical relation (Formula IV) disclosed in U.S. Pat. No. 5,705,399, for determining concentration of the predetermined osteoarthritis biomarker in the urine sample. The mathematical relation is described as follows:

$$Δ f = \frac{- 2 {f}_{0}^{2} Δ m}{A}$$

where Δf is the frequency shift, in Hz, associated with mass of the antigen captured by the antibody, f₀ is the fundamental operating frequency of the quartz resonator 121, in MHz, Am is the change in mass on the first side 121a (i.e., the sample contacting side), and A is the surface area of the sample contacting side, in cm².

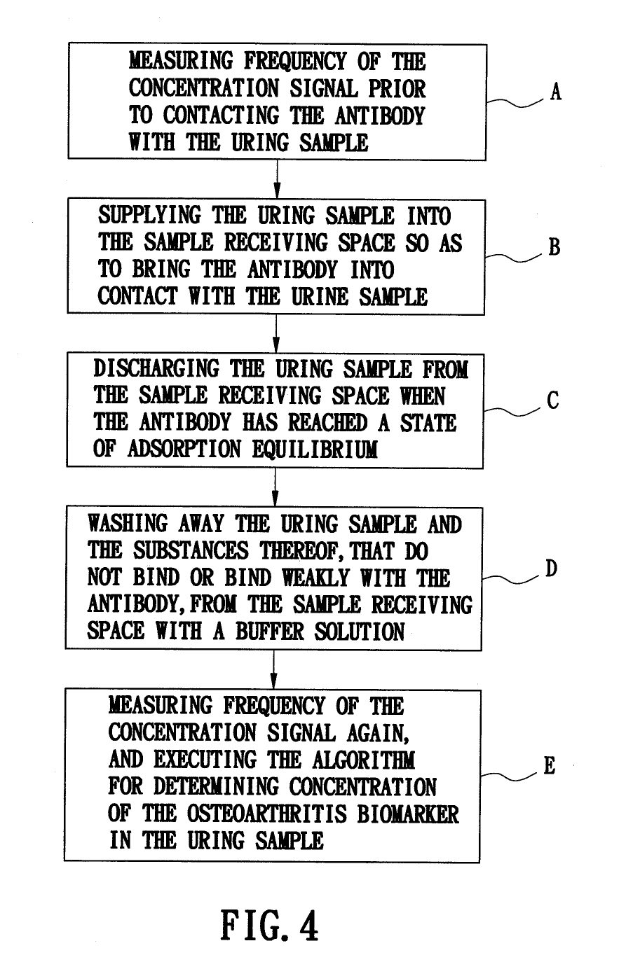

Referring to FIG. 4, operation of the system described hereinabove according to the present invention includes the steps of:
a) configuring the processing unit 132 to measure frequency of the concentration signal prior to contacting the antibody with the urine sample;
b) operating the first flow control unit 111a to supply the urine sample into the sample receiving space 112e so as to bring the antibody into contact with the urine sample;
c) when the antibody has reached a state of adsorption equilibrium, operating the second flow control unit 111b such that the urine sample is able to be discharged from the sample receiving space 112e through the second channel 112b and collected in a vessel 111c;
d) injecting a buffer solution having a pH value that ranges between 7 and 8 from the first channel 112a to wash away the urine sample and the substances thereof, that do not bind or bind weakly with the antibody, from the sample receiving space 112e, through the second channel 112b; and
e) configuring the processing unit 132 to measure frequency of the concentration signal again, and executing the aforesaid algorithm for determining concentration of the osteoarthritis biomarker in the urine sample.

In summary, the system for determining concentration of a predetermined osteoarthritis biomarker in a urine sample, according to the present invention, has a modular design, only requires a few operating steps and short diagnosis time, does not require pre-processing of the urine sample, and therefore is suitable for implementation as a portable device at a relatively low cost.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A system for determining concentration of a predetermined osteoarthritis biomarker in a urine sample, said system comprising:
a flow sensor assembly including
a quartz crystal microbalance (QCM) sensor device including a quartz resonator and an antibody applied to said quartz resonator and capable of binding with the predetermined osteoarthritis biomarker in the urine sample, said QCM sensor device having a sample contacting side and generating a concentration signal corresponding to mass of the predetermined osteoarthritis biomarker that binds to said antibody, and
a flow cell device including a first cell element that has a sensor confronting side formed with a recess portion, and a first limiting element that is disposed at said sensor confronting side of said first cell element, that is disposed to surround said recess portion of said first cell element, that contacts said sample contacting side of said QCM sensor device, and that cooperates with said recess portion of said first cell element and said sample contacting side of said QCM sensor device to confine a sample receiving space for receiving the urine sample; and
a monitoring device coupled to said QCM sensor device for receiving the concentration signal therefrom, and operable to determine concentration of the predetermined osteoarthritis biomarker in the urine sample using the concentration signal.

2. The system as claimed in claim 1, wherein said quartz resonator of said QCM sensor device includes:
a quartz body having a first side that serves as said sample contacting side of said QCM sensor device, and a second side that is opposite to said first side; and
first and second electrodes disposed respectively on said first and second sides of said quartz body;
said antibody being applied to said first electrode.

3. The system as claimed in claim 1, wherein said quartz resonator of said QCM sensor device generates a frequency signal corresponding to the mass of the predetermined osteoarthritis biomarker that binds to said antibody, said QCM sensor device further including a feedback oscillator unit coupled electrically to said quartz resonator for receiving the frequency signal therefrom and for generating the concentration signal corresponding to the frequency signal.

4. The system as claimed in claim 3, wherein said feedback oscillator unit is operable to amplify the frequency signal to generate the concentration signal having frequency and phase characteristics substantially identical to those of the frequency signal.

5. The system as claimed in claim 1, wherein said antibody is selected from the group consisting of an anti-type II collagen neoepitope (TIINE), an anti-cartilage oligometric matrix protein (COMP), a neoepitope at the C-terminus of the ¾ length type II collagen cleavage product created by the cleavage of type II collagen by collagenases, and combinations thereof.

6. The system as claimed in claim 1, wherein said flow cell device of said flow sensor assembly further includes a second cell element for securing separably said QCM sensor device to said first cell element.

7. The system as claimed in claim 6, wherein said second cell element is disposed at said sensor confronting side of said first cell element and cooperates with said first cell element to form a sensor disposing space within which said QCM sensor device is disposed.

8. The system as claimed in claim 7, wherein said flow cell device of said flow sensor assembly further includes a second limiting element disposed between said QCM sensor device and said second cell element.

9. The system as claimed in claim 8, wherein each of said first and second limiting elements is a waterproofing ring made of plastic.

10. The system as claimed in claim 1, wherein said first cell element is formed with first and second channels, each of which is in fluid communication with said recess portion.

11. The system as claimed in claim 10, wherein said flow cell device further includes first and second flow control units coupled to said first cell element for controlling flow of the urine sample through a respective one of said first and second channels.

12. The system as claimed in claim 11, wherein said first flow control unit and said first channel cooperate to form a supply path for introducing the urine sample into said sample receiving space, and said second flow control unit and said second channel cooperate to form a discharge path for discharging the urine sample from said sample receiving space.

13. A flow cell device adapted for use with a sensor device to detect a liquid sample, comprising:
a first cell element that has a sensor confronting side formed with a recess portion; and
a first limiting element that is disposed at said sensor confronting side of said first cell element, that is disposed to surround said recess portion of said first cell element, and that is adapted to contact a sample contacting side of the sensor device;
said first limiting element, together with said recess portion of said first cell element, being adapted to cooperate with the sample contacting side of the sensor device to confine a sample receiving space for receiving the liquid sample.

14. The flow cell device as claimed in claim 13, further comprising a second cell element adapted for securing separably the sensor device to said first cell element.

15. The flow cell device as claimed in claim 14, wherein said second cell element is disposed at said sensor confronting side of said first cell element and cooperates with said first cell element to form a sensor disposing space within which the sensor device is to be disposed.

16. The flow cell device claimed in claim 15, further comprising a second limiting element adapted to be disposed between the sensor device and said second cell element.

17. The flow cell device as claimed in claim 16, wherein each of said first and second limiting elements is a waterproofing ring made of plastic.

18. The flow cell device as claimed in claim 13, wherein said first cell element is formed with first and second channels, each of which is in fluid communication with said recess portion.

19. The flow cell device as claimed in claim 18, further comprising first and second flow control units coupled to said first cell element for controlling flow of the liquid sample through a respective one of said first and second channels.

20. The flow cell device as claimed in claim 19, wherein said first flow control unit and said first channel cooperate to form a supply path for introducing the liquid sample into said sample receiving space, and said second flow control unit and said second channel cooperate to forma discharge path for discharging the liquid sample from said sample receiving space.

21. A method for determining concentration of a predetermined osteoarthritis biomarker in a urine sample, said method comprising the steps of:
a) providing a flow sensor assembly which comprises a quartz crystal microbalance (QCM) sensor device and a flow cell device,
the QCM sensor device including a quartz resonator and an antibody applied to the quartz resonator and capable of binding with the predetermined osteoarthritis biomarker in the urine sample, the QCM sensor device having a sample contacting side and generating a concentration signal corresponding to mass of the predetermined osteoarthritis biomarker that binds to the antibody,
the flow cell device including a cell element that has a sensor confronting side formed with a recess portion, and a limiting element that is disposed at the sensor confronting side of the cell element, that is disposed to surround the recess portion of the cell element, that contacts the sample contacting side of the QCM sensor device, and that cooperates with the recess portion of the cell element and the sample contacting side of the QCM sensor device to confine a sample receiving space for receiving the urine sample;
b) with the use of a monitoring device coupled to the QCM sensor device, measuring a frequency response of the QCM sensor device prior to contacting the QCM sensor device with the urine sample;
c) supplying the urine sample into the sample receiving space so as to bring the sample contacting side of the QCM sensor device into contact with the urine sample;
d) removing the urine sample from the sample receiving space and supplying a buffer solution into the sample receiving space;
e) with the use of the monitoring device, measuring a frequency response of the QCM sensor device from the concentration signal after supplying the buffer solution into the sample receiving space; and
f) configuring the monitoring device to determine the concentration of the predetermined osteoarthritis biomarker in the urine sample with reference to the measured frequency responses obtained in steps b) and e).

22. The method as claimed in claim 21, wherein the buffer solution has a pH value ranging between 7 and 8.
